(19) United States
(12) Patent Application Publication
(10) Pub. No.: US 2026/0219252 A1
(43) Pub. Date: Jul. 30, 2026

(54) OIL-AND-FAT DETERIORATION PREDICTION DEVICE, OIL-AND-FAT DETERIORATION PREDICTION SYSTEM, AND OIL-AND-FAT DETERIORATION PREDICTION METHOD

(71) Applicant: J-OIL MILLS, Inc., Tokyo (JP)

(72) Inventors: Kenichi KAKIMOTO, Tokyo (JP); Ryohei WATANABE, Tokyo (JP); Eri NUTAHARA, Tokyo (JP); Moeka ONO, Tokyo (JP)

(21) Appl. No.: 19/146,854

(22) PCT Filed: Dec. 8, 2023
(86) PCT No.: PCT/JP2023/044062

§ 371 Date: Jul. 9, 2025

(30) Foreign Application Priority Data
Jan. 17, 2023 (JP) 2023-005156

(51) Int. Cl. G01N 33/03

(52) U.S. Cl. CPC G01N 33/03

(57) ABSTRACT

A server 7 for predicting deterioration of frying oil P, comprises: a storage section 72 configured to store a deterioration characteristic model a deterioration indicator of the frying oil P and a deterioration reference value of the frying oil P; a data acquisition section 71 configured to acquire a measured value of the deterioration indicator; a model update section 73 configured to update the deterioration characteristic model based on a plurality of most recent measured values a deterioration prediction section 74 configured to predict deterioration information of the frying oil P, based on a latest measured value the deterioration characteristic model and the deterioration reference value stored; and a result output section 75 configured to output a result of prediction of the deterioration information.

### TECHNICAL FIELD

The present invention relates to an oil and fat deterioration predication device, an oil and fat deterioration prediction system, and an oil and fat deterioration prediction method, which are provided to predict deterioration of oils and fats.

### BACKGROUND ART

Oils and fats, in general, deteriorate as they are exposed to air (oxygen) for longer periods of time. Therefore, in stores and factories that use oils and fats, the degree of deterioration (hereinafter, referred to as “deterioration degree”) of the oils and fats is monitored using appropriate indicators, so that oils and fats that have reached an oil disposal time point can be disposed and replaced with fresh ones. Continuing to use oils and fats that have reached the oil disposal time point may cause problems such as, in the case where they are edible oils, impaired flavor and deterioration in the quality of ingredients, and in the case where they are industrial oils, malfunctions and reduced lifespan of equipment. For these problems, prediction of the deterioration of oils and fats may have been carried out to prevent the optimal timing for disposing of the oils and fats from being missed.

For example, Patent Literature 1 discloses a method of predicting the remaining service life of mineral oils, using a first curve as a future deterioration curve if a difference between a deterioration degree corresponding to light transmittance of the mineral oils at a given time point and a deterioration degree predicted based on a deterioration curve at that time point is less than a predetermined value while using a second curve, which shows faster deterioration than that according to the first curve, as the future deterioration curve if the difference between the deterioration degree corresponding to the light transmittance of the mineral oils at the given time point and the deterioration degree predicted based on the deterioration curve at that time point is more than the predetermined value.

According to the conventional method described above, for example, if the moisture content of mineral oils increases and thus the deterioration accelerates, using the second curve, which shows faster deterioration than that according to the first curve, as the future deterioration curve, enables accurate prediction of the remaining service life of the mineral oils based on its usage environment.

### CITATION LIST

### Patent Literature

Patent Literature 1: JP-A-2019-219281

### SUMMARY OF INVENTION

### Technical Problem

Oils and fats which are the targets of the deterioration prediction, especially if they are edible oils, frequently changes in the deterioration speed depending on factors such as the type and quantity of ingredients to be cooked and the frequency of cooking. Therefore, even if updating an edible oil deterioration prediction model (future deterioration curve) by the method according to Patent Literature 1, only the deviation of the actual deterioration degree from the predicted deterioration degree at a given time point is considered, which makes the accuracy of the prediction insufficient.

An object of the present invention is to provide an oil and fat deterioration predication device, an oil and fat deterioration prediction system, and an oil and fat deterioration prediction method, which are capable of predicting the deterioration of oils and fats with high accuracy based on change in the usage environment and usage situation of the oils and fats.

### Solution to Problem

[1] The present invention provides an oil and fat deterioration prediction device for predicting deterioration of oil and fat, comprising: a storage section configured to store a deterioration characteristic model and a deterioration reference value, the deterioration characteristic model being indicative of transition of a deterioration indicator that is an indicator indicative of a deterioration degree of the oil and fat, the deterioration reference value being indicative of a reference related to the deterioration indicator; a data acquisition section configured to acquire a measured value of the deterioration indicator; a model update section configured to update the deterioration characteristic model stored in the storage section based on a plurality of most recent measured values acquired by the data acquisition section; a deterioration prediction section configured to predict deterioration information that is information about deterioration of the oil and fat, based on a latest measured value acquired by the data acquisition section, the deterioration characteristic model updated by the model update section, and the deterioration reference value stored in the storage section; and a result output section configured to output a result of prediction of the deterioration information predicted by the deterioration prediction section.

[2] Preferably, in the oil and fat deterioration predication device according to [1] above, the oil and fat is edible oil to be used in cooking of an ingredient using a cooking tool, and the deterioration information includes at least one of: data indicative of a remaining time until the edible oil reaches a predetermined oil disposal time point; data indicative of a timing of performing a removing/adding oil operation, in which a part of the edible oil in the cooking tool is removed and another edible oil that is different from the edible oil is added therein; data indicative of a timing of filtering the edible oil in the cooking tool; or data indicative of a remaining amount of ingredients that can be cooked until the edible oil reaches a predetermined deterioration degree.

[3] Preferably, in the oil and fat deterioration predication device according to [2] above, the deterioration characteristic model includes at least one of: a model indicative of a correlation between a heating time of the edible oil and the deterioration indicator; or a model indicative of a correlation between an amount of ingredients that can be cooked using the edible oil and the deterioration indicator.

[4] Preferably, in the oil and fat deterioration predication device according to [1] above, the data acquisition section acquires the measured value of the deterioration indicator at predetermined intervals, and the model update section updates the deterioration characteristic model stored in the storage section based on measured values for most recent two operating days of a store that uses the oil and fat, including the latest measured value acquired by the data acquisition section.

[5] Furthermore, the present invention provides an oil and fat deterioration prediction system for predicting deterioration of oil and fat and notifying a result of prediction, the system comprising: a measurement device for measuring a deterioration indicator that is an indicator indicative of a deterioration degree of the oil and fat; an oil and fat deterioration prediction device for predicting deterioration information that is information about the deterioration of the oil and fat, using a measured value of the deterioration indicator measured by the measurement device; and a notification device for notifying the result of prediction of the deterioration information predicted by the oil and fat deterioration prediction device, the oil and fat deterioration prediction device being configured to: store a deterioration characteristic model indicative of transition of the deterioration indicator and a deterioration reference value indicative of a reference related to the deterioration indicator; acquire the measured value of the deterioration indicator measured by the measurement device; update the deterioration characteristic model as stored based on a plurality of most recent measured values as acquired; predict the deterioration information based on a latest measured value as acquired, the deterioration characteristic model as updated, and the deterioration reference value as stored; and output the result of prediction of the deterioration information as predicted to the notification device.

[6] Preferably, in the oil and fat deterioration prediction system according to [5] above, the oil and fat is edible oil to be used in cooking of an ingredient using a cooking tool, and the deterioration information includes at least one of: data indicative of a remaining time until the edible oil reaches a predetermined oil disposal time point; data indicative of a timing of performing a removing/adding oil operation, in which a part of the edible oil in the cooking tool is removed and another edible oil that is different from the edible oil is added therein; data indicative of a timing of filtering the edible oil in the cooking tool; or data indicative of a remaining amount of ingredients that can be cooked until the edible oil reaches a predetermined deterioration degree.

[7] Preferably, in the oil and fat deterioration prediction system according to [6] above, the deterioration characteristic model includes at least one of: a model indicative of a correlation between a heating time of the edible oil and the deterioration indicator; or a model indicative of a correlation between an amount of ingredients that can be cooked using the edible oil and the deterioration indicator.

[8] Preferably, in the oil and fat deterioration prediction system according to [5] above, the oil and fat deterioration prediction device is configured to: acquire the measured value of the deterioration indicator at predetermined intervals, and update the deterioration characteristic model as stored, based on measured values for most recent two operating days of a store that uses the oil and fat, including the latest measured value as acquired.

[9] Still further, the present invention provides an oil and fat deterioration prediction method for predicting deterioration of oil and fat and notifying a result of prediction, using a measurement device for measuring a deterioration indicator that is an indicator indicative of a deterioration degree of the oil and fat, an oil and fat deterioration prediction device for predicting deterioration information that is information about the deterioration of the oil and fat using a measured value of the deterioration indicator measured by the measurement device, and a notification device for notifying the result of prediction of the deterioration information predicted by the oil and fat deterioration prediction device, the oil and fat deterioration prediction device being configured to store a deterioration characteristic model indicative of transition of the deterioration indicator and a deterioration reference value indicative of a reference related to the deterioration indicator, the method comprising: a measuring step of measuring the deterioration indicator, by the measurement device; a data acquiring step of acquiring the measured value measured in the measuring step, by the oil and fat deterioration prediction device; a model updating step of updating the deterioration characteristic model as stored based on a plurality of most recent measured values acquired in the data acquiring step, by the oil and fat deterioration prediction device; a deterioration predicting step of predicting the deterioration information based on a latest measured value acquired in the data acquiring step, the deterioration characteristic model updated in the model updating step, and the deterioration reference value as stored, by the oil and fat deterioration prediction device; a result outputting step of outputting the result of prediction predicted in the deterioration predicting step to the notification device, by the oil and fat deterioration prediction device; and a notifying step of acquiring and notifying the result of prediction of the deterioration information output in the result outputting step, by the notification device.

Preferably, in the oil and fat deterioration prediction method according to [9] above, the oil and fat is edible oil to be used in cooking of an ingredient using a cooking tool, and the deterioration information includes at least one of: data indicative of a remaining time until the edible oil reaches a predetermined oil disposal time point; data indicative of a timing of performing a removing/adding oil operation, in which a part of the edible oil in the cooking tool is removed and another edible oil that is different from the edible oil is added therein; data indicative of a timing of filtering the edible oil in the cooking tool; or data indicative of a remaining amount of ingredients that can be cooked until the edible oil reaches a predetermined deterioration degree.

[11] Preferably, in the oil and fat deterioration prediction method according to above, the deterioration characteristic model includes at least one of: a model indicative of a correlation between a heating time of the edible oil and the deterioration indicator; or a model indicative of a correlation between an amount of ingredients that can be cooked using the edible oil and the deterioration indicator.

[12] Preferably, in the oil and fat deterioration prediction method according to [9] above, in the data acquiring step, the oil and fat deterioration prediction device acquires the measured value of the deterioration indicator at predetermined intervals, and in the model updating step, the oil and fat deterioration prediction device updates the deterioration characteristic model as stored based on measured values for most recent two operating days of a store that uses the oil and fat, including the latest measured value acquired in the data acquiring step.

### Advantageous Effects of Invention

According to the present invention, it is possible to provide an oil and fat deterioration predication device, an oil and fat deterioration prediction system, and an oil and fat deterioration prediction method, which are capable of predicting the deterioration of oils and fats with high accuracy based on change in the usage environment and usage situation of the oils and fats. The problems, configurations, and advantageous effects other than those described above will be clarified by explanation of the embodiments below.

### BRIEF DESCRIPTION OF DRAWINGS

FIG. 1 illustrates a part of a cooking area in which deep-frying is performed.

FIG. 2 illustrates how an acid value of frying oil changes relative to a heating time of the frying oil at a store.

FIG. 3 illustrates how a color of frying oil changes relative to a heating time of the frying oil at a store.

FIG. 4 illustrates how an acid value of frying oil changes relative to the number of pieces of a deep-frying material cooked using the frying oil at a store.

FIG. 5 illustrates how a color of frying oil changes relative to the number of pieces of a deep-frying material cooked using the frying oil at a store.

FIG. 6 is a system configuration diagram illustrating a configuration example of a frying oil deterioration prediction system.

FIG. 7 illustrates an example of a hardware configuration of a server.

FIG. 8 is a functional block diagram illustrating functions provided in a server.

FIG. 9 illustrates a flowchart of a flow of processing to be executed in a server.

FIG. 10 illustrates an example of a deterioration characteristic model when a heating time of frying oil is 40 hours.

FIG. 11 illustrates an update image of a deterioration characteristic model when a heating time of frying oil is 48 hours.

FIG. 12 illustrates an update image of a deterioration characteristic model when a heating time of frying oil is 56 hours.

FIG. 13 illustrates an update image of a deterioration characteristic model when a heating time of frying oil is 64 hours.

FIG. 14 illustrates how an acid value of frying oil changes relative to a heating time of the frying oil in a supermarket store.

FIG. 15 illustrates a table for the graph illustrated in FIG. 14, in which the number of pieces of data used for a deterioration characteristic model, a period of time corresponding to the number of pieces of data, the time required to reach an oil disposal time point from the current time, an error, and the rate of error are listed.

FIG. 16 illustrates how an acid value of frying oil changes relative to a heating time of the frying oil in a convenience store.

FIG. 17 illustrates a table for the graph illustrated in FIG. 16, in which the number of pieces of data used for a deterioration characteristic model, a period of time corresponding to the number of pieces of data, the time required to reach an oil disposal time point from the current time, an error, and the rate of error are listed.

FIG. 18 illustrates a graph indicative of change in an acid value of frying oil relative of the number of operating days of a store.

### DESCRIPTION OF EMBODIMENTS

Hereinafter, as an aspect of an oil and fat deterioration prediction system according to embodiments of the present invention, a deterioration prediction system applicable to cooking of fried foods such as fried chicken, croquettes, and karaage, using edible oils in, for example, convenience stores and supermarkets.

Cooking of fried foods is referred herein as “deep-frying”, edible oil to be used in deep-frying is referred herein as “frying oil”, and ingredients to be deep fried is referred herein as “deep-frying material”.

### (Arrangement in Cooking Area 1)

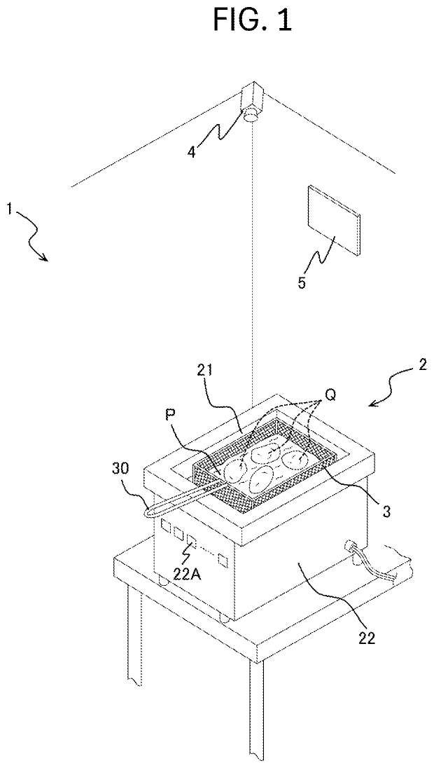

Firstly, an example of an environment in which deep-frying is performed will be described with reference to FIG. 1.

FIG. 1 illustrates a part of a cooking area 1 in which deep-frying is performed.

For example, in a store such as a convenience store or a supermarket, the cooking area 1 in which deep-frying is performed is provided in the store so as to provide customers with freshly deep-fried foods. Within the cooking area 1, as a cooking tool to be used in deep-frying, for example, an electric fryer 2 is installed.

The fryer 2 includes an oil vat 21 for holding frying oil P therein, and a housing 22 for accommodating the oil vat 21. On a side surface of the housing 22, a plurality of switches 22A for setting the temperature of the frying oil P and the details of the deep-frying for each type of deep-frying materials Q is provided.

For performing deep-frying, firstly, a cook places the deep-frying material Q in a fry basket 3 having a handle 30, and then hooks the handle 30 on an upper end portion of the housing 22 so as to immerse, in the frying oil P, the deep-frying material Q placed in the fry basket 3. At the same time, therebefore, or thereafter, the cook operates one of the switches 22A which corresponds to the type of the deep-frying material Q which is to be cooked.

Subsequently, the fryer 2 identifies the one of the switches 22A which was operated by the cook, and when a deep-frying time, which is associated with the operated one of the switches 22A, passes, the fryer 2 notifies the cook of the completion of deep-frying. At the same time, the fry basket 3 holding the deep-fried food (deep-frying material Q after being deep fried) automatically rises from the oil vat 21 so that the deep-fried food that has been immersed in the frying oil P is pulled up.

As a technique of informing the completion of deep-frying of a fried food, for example, a buzzer sound may be output from a speaker of the fryer 2 or completion of deep-frying may be shown on a monitor installed near the fryer 2.

The cook who has noticed the completion of deep-frying pulls up the fry basket 3 to take the fried food out therefrom. The operation of pulling up the fry basket 3 from the oil vat 21 may be automatically performed by a drive mechanism which can be provided in the fryer 2.

In the present embodiment, the cooking area 1 includes a camera 4 for acquiring an image of the surface of the frying oil P in the oil vat 21, which is mounted to the ceiling which is positioned above the oil vat 21. However, the camera 4 does not necessarily have to be mounted to the ceiling positioned above the oil vat 21. The camera 4 may be mounted to any position, for example, a wall near the fryer 2, as long as it is held at a position allowing the image of the surface of the frying oil P in the oil vat 21 to be captured.

The camera 4 is a video camera for capturing a video or a still camera for capturing a still image, and is used in measurement of the color of the frying oil P. Specifically, the color of the frying oil P is measured based on a brightness value (for example, RGB value) of an image of the surface of the frying oil P in the oil vat 21 captured by the camera 4. Thus, the image captured by the camera 4 needs to include at least the image of the surface of the frying oil P in the oil vat 21, however, it may include an image other than the image of the surface of the frying oil P, such as an image of a portion of the oil vat 21 or an image of an object (specifically, the deep-frying material Q or a portion of the fry basket 3) immersed in the frying oil P.

The color of the frying oil P gets darker as the heating time progresses, and accordingly, it is used as a deterioration indicator which is an indicator indicative of the degree of deterioration of the frying oil P (hereinafter, simply referred to as “deterioration degree”). The deterioration indicators of the frying oil P other than the color of the frying oil P are, for example, the acid value (AV) of the frying oil P, the amount of polar compounds (PC) of the frying oil P, the viscosity of the frying oil P, the rate of increase in viscosity of the frying oil P, the anisidine value of the frying oil P, the carbonyl value of the frying oil P, the smoke point of the frying oil P, the tocopherol contents of the frying oil P, the iodine value of the frying oil P, the refractive indicator of the frying oil P, the amount of volatile components of the frying oil P, the volatile component composition of the frying oil P, the flavor of the frying oil P, the amount of volatile components of a fried food obtained by deep-frying using the frying oil P, the volatile component composition of a fried food obtained by deep-frying using the frying oil P, and the flavor of a fried food obtained by deep-frying using the frying oil P.

A user who uses the frying oil P (for example, a cook, a store staff, or the like) measures the deterioration indicator of the frying oil P, and based on a measured value of the deterioration indicator of the frying oil P, determines or predicts the deterioration of the frying oil P, so that the quality of the frying oil P and the qualities of the fried foods obtained by deep-frying using the frying oil P can be maintained.

Each of the deterioration indicators of the frying oil P can be measured using a measurement device such as the camera 4 or various sensors. For example, for measurement of the acid value (AV) of the frying oil P, a test paper for measuring the acid value based on the change in color of a portion on which the frying oil P is dropped, a measurement apparatus which is to be immersed in the frying oil P so as to directly measure the acid value of the frying oil P, or the like may be used. For measurement of the amount of polar compounds (PC) of the frying oil P, a measurement apparatus which is to be immersed in the frying oil P so as to directly measure the amount of polar compounds contained in the frying oil P or the like may be used. For measurement of the amount of volatile components and volatile component composition of the frying oil P and measurement of the amount of volatile components and volatile component composition of the fried food obtained by deep-frying using the frying oil P, a commonly-used gas sensor (for example, semiconductor gas sensor or crystal oscillator gas sensor) or the like may be used.

The techniques of measuring the deterioration indicators other than the ones mentioned above include, for example, a technique of recognizing an image captured by the camera 4 by means of an image recognition technique to identify the type and number of fried foods obtained by deep-frying using the frying oil P, and predicting each deterioration indicator based on the correlation between the type and number of fried foods and each deterioration indicator, a technique of measuring the spectrum of the frying oil P by means of a spectrometer and predicting each deterioration indicator based on the correlation between the spectrum of the frying oil P and each deterioration indicator, and a technique of predicting each deterioration indicator based on the correlation between the number of times the setting switches 22A of the fryer 2 have been operated (that is, the number of times the fried foods have been cooked) and each deterioration indicator.

A measurement device and measurement technique for measuring the deterioration indicator of the frying oil P are not necessarily limited to the ones described above, but other known measurement devices and measurement techniques may be used.

### (Deterioration Characteristic of Frying Oil P)

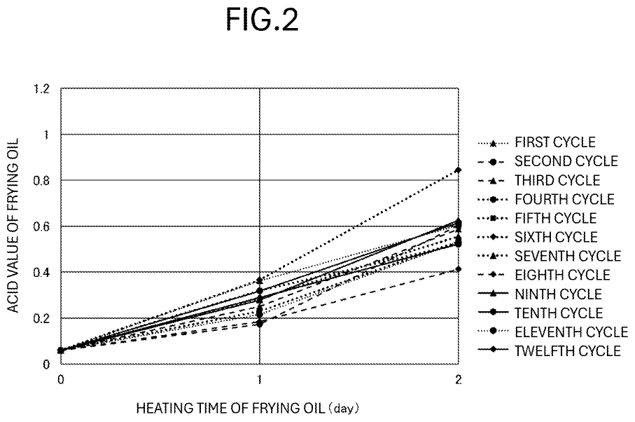

Next, referring to FIG. 2 to FIG. 5, a deterioration characteristic of the frying oil P will be described.

The frying oil P has a deterioration characteristic in which it deteriorates as the heating time thereof or the number of pieces of the deep-frying material Q to be deep-fried therewith increases. This deterioration characteristic of the frying oil P is not limited to one, but varies depending on the usage environment and situations of the frying oil P. Therefore, for example, each of the frying oils P, even if being used within the same store, may not always have the same deterioration characteristics.

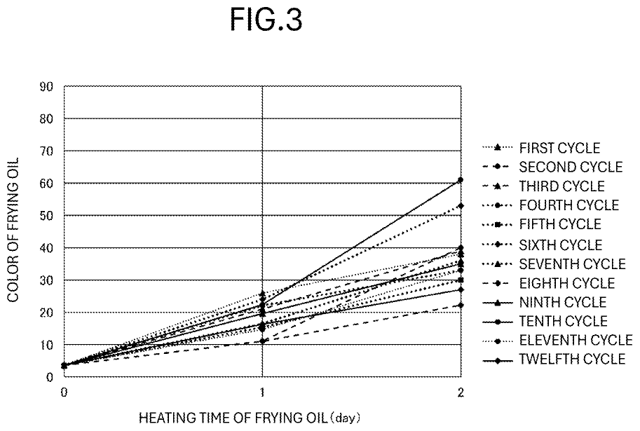

FIG. 2 illustrates a graph showing how the acid value of the frying oil P changes relative to the heating time of the frying oil P at a store. FIG. 3 illustrates a graph showing how the color of the frying oil P changes relative to the heating time of the frying oil P at a store.

FIG. 2 illustrates how the acid value of the frying oil P changes relative to the heating time [day] of the frying oil P for each of the first to twelfth cycles at a store. Here, one “cycle” represents the period from when the frying oil P is in a fresh condition to when it is disposed. FIG. 3 illustrates how the color of the frying oil P changes relative to the heating time [day] of the frying oil P for each of the first to twelfth cycles at a store.

Each of FIG. 2 and FIG. 3 illustrates only the heating time of the frying oil P for two days. However, the frying oil P is not necessarily disposed every two days, and may continue to be used beyond the third day. For example, in the case where the frying oil P continues to be used for four days, FIG. 2 would illustrate how the frying oil P changes in the acid value until the heating time of the frying oil P reaches the second day from its fresh condition and FIG. 3 would illustrate how the frying oil P changes in the color until the heating time of the frying oil P reaches the second day from its fresh condition.

Each of FIG. 2 and FIG. 3 illustrates the first cycle with a graph in which ▴ marks are connected to each other by a dotted line, the second cycle with a graph in which • marks are connected to each other by a dashed line, the third cycle with a graph in which ▴ marks are connected to each other by a dashed line, the fourth cycle with a graph in which • marks are connected to each other by a dotted line, the fifth cycle with a graph in which ▪ marks are connected to each other by a dotted line, the sixth cycle with a graph in which ♦ marks are connected to each other by a dotted line, the seventh cycle with a graph in which ▴ marks are connected to each other by a dotted line, the eighth cycle with a graph in which ♦ marks are connected to each other by a dashed line, the ninth cycle with a graph in which ▴ marks are connected to each other by a solid line, the tenth cycle with a graph in which • marks are connected to each other by a solid line, the eleventh cycle with a graph in which • marks are connected to each other by a dotted line, and the twelfth cycle with a graph in which ♦ marks are connected to each other by a solid line.

As illustrated in FIG. 2, for example, the acid value of the frying oil P in the sixth cycle after one day passed from the fresh oil condition of the frying oil P is approximately 0.38, and the acid value thereof after two days passed from the fresh oil condition of the frying oil P is approximately 0.85. On the other hand, the acid value of the frying oil P in the eighth cycle after one day passed from the fresh oil condition of the frying oil P is approximately 0.19, and the acid value thereof after two days passed from the fresh oil condition of the frying oil P is approximately 0.41. Among the frying oils P of the other first to fifth cycles, the seventh cycle, and the ninth to twelfth cycles as well, how the acid value thereof increases relative to the heating time is different from each other.

As illustrated in FIG. 3, for example, the color of the frying oil P in the tenth cycle after one day passed from the fresh oil condition of the frying oil P is approximately 23, and the color thereof after two days passed from the fresh oil condition of the frying oil P is approximately 61. On the other hand, the color of the frying oil P in the twelfth cycle after one day passed from the fresh oil condition of the frying oil P is approximately 16, and the color thereof after two days passed from the fresh oil condition of the frying oil P is approximately 27. Among the frying oils P of the first to ninth cycles and the eleventh cycle as well, how the color thereof increases relative to the heating time is different from each other.

Thus, even in the case of the frying oils P used within the same store, how the deterioration indicators (acid value in FIG. 2 and color in FIG. 3) change relative to the heating time is different among the cycles.

How the deterioration indicator of the frying oil P changes relative to the heating time of the frying oil P is shown in each of FIG. 2 and FIG. 3, however, it is not limited thereto, and how the deterioration indicator of the frying oil P changes relative to the number of pieces of the deep-frying material Q which can be cooked using the frying oil P is different among the cycles in the same manner.

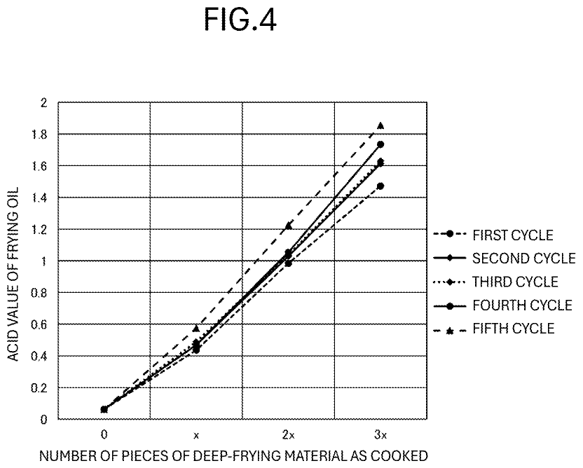

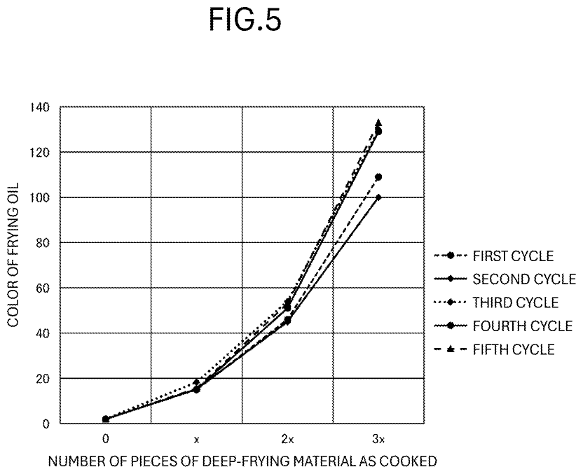

FIG. 4 illustrates a graph showing how the acid value of the frying oil P changes relative to the number of pieces of the deep-frying material Q cooked using the frying oil P at a store. FIG. 5 illustrates a graph showing how the color of the frying oil P changes relative to the number of pieces of the deep-frying material Q cooked using the frying oil P at a store.

FIG. 4 illustrates how the acid value of the frying oil P changes relative to the number of pieces of the deep-frying material Q to be deep-fried using the frying oil P in each of the first to fifth cycles at a store. Here, one “cycle” represents the period from when the frying oil P is in a fresh condition to when it is disposed. FIG. 5 illustrates how the color of the frying oil P changes relative to the number of pieces of the deep-frying material Q to be deep-fried using the frying oil P in each of the first to fifth cycles at a store.

Here, in each of FIG. 4 and FIG. 5, the sign “x” provided on the horizontal axis represents the number of pieces of the deep-frying material Q for one set, which is predetermined at each store. For example, it is predetermined that, at a store that sells only croquettes, one set includes 10 croquettes, and at a store that sells two types of products which are croquettes and fried chicken, one set includes 4 croquettes and 5 pieces of fried chicken. By checking the number of sets, the deterioration of the frying oil P can be managed.

Each of FIG. 4 and FIG. 5 illustrates the case where the number of pieces of the deep-frying material Q to be deep-fried using the frying oil P is up to 3× pieces (three sets). However, the frying oil P is not necessarily disposed once every time the 3× pieces of the deep-frying material Q are cooked, and may continue to be used without being disposed even when more than 4× pieces (four sets) of the deep-frying material Q are cooked. For example, in the case where the frying oil P continues to be used until 6× pieces of the deep-frying material Q are cooked, FIG. 4 would illustrate how the frying oil P changes in the acid value until the number of pieces of the deep-frying material Q to be deep-fried using the frying oil P reaches 3× pieces from the fresh condition of the frying oil P, within the period in which the number of pieces deep-fried reaches 6× pieces, and FIG. 5 would illustrate how the frying oil P changes in the color until the number of pieces of the deep-frying material Q to be deep-fried using the frying oil P reaches 3× pieces from the fresh condition of the frying oil P, within the period in which the number of pieces deep-fried reaches 6× pieces.

Each of FIG. 4 and FIG. 5 illustrates the first cycle with a graph in which • marks are connected to each other by a dashed line, the second cycle with a graph in which ♦ marks are connected to each other by a solid line, the third cycle with a graph in which ♦ marks are connected to each other by a dotted line, the fourth cycle with a graph in which • marks are connected to each other by a solid line, and the fifth cycle with a graph in which ▴ marks are connected to each other by a dashed line.

As illustrated in FIG. 4, for example, the acid value of the frying oil P in the first cycle after x pieces (one set) of the deep-frying material Q are deep-fried in the fresh oil condition of the frying oil P is approximately 0.41, the acid value thereof after 2× pieces (two sets) of the deep-frying material Q are deep-fried in the fresh oil condition of the frying oil P is approximately 0.99, and the acid value thereof after 3× pieces (three sets) of the deep-frying material Q are deep-fried in the fresh oil condition of the frying oil P is approximately 1.48. On the other hand, the acid value of the frying oil P in the fifth cycle after x pieces of the deep-frying material Q are deep-fried in the fresh oil condition of the frying oil P is approximately 0.58, the acid value thereof after 2x pieces of the deep-frying material Q are deep-fried in the fresh oil condition of the frying oil P is approximately 1.21, and the acid value thereof after 3x pieces of the deep-frying material Q are deep-fried in the fresh oil condition of the frying oil P is approximately 1.82. Among the frying oils P of the other second to fourth cycles as well, how the acid value thereof increases relative to the number of pieces of the deep-frying material Q cooked using the frying oil P is different from each other.

As illustrated in FIG. 5, for example, the color of the frying oil P in the second cycle after x pieces of the deep-frying material Q are deep-fried in the fresh oil condition of the frying oil P is approximately 16, the color thereof after 2× pieces of the deep-frying material Q are deep-fried in the fresh oil condition of the frying oil P is approximately 45, and the color thereof after 3× pieces of the deep-frying material Q are deep-fried in the fresh oil condition of the frying oil P is just 100. On the other hand, the color of the frying oil P in the fifth cycle after x pieces of the deep-frying material Q are deep-fried in the fresh oil condition of the frying oil P is approximately 19, the color thereof after 2× pieces of the deep-frying material Q are deep-fried in the fresh oil condition of the frying oil P is approximately 50, and the color thereof after 3× pieces of the deep-frying material Q are deep-fried in the fresh oil condition of the frying oil P is approximately 132. Among the frying oils P of the other first, third, and fourth cycles as well, how the color thereof increases relative to the number of pieces of the deep-frying material Q cooked using the frying oil P is different from each other.

Thus, even among the frying oils P used within the same store, how the deterioration indicators (acid value in FIG. 4 and color in FIG. 5) change relative to the number of pieces of the deep-frying material Q cooked using the frying oil P is different in each cycle.

### (Configuration of Frying Oil Deterioration Prediction System 100)

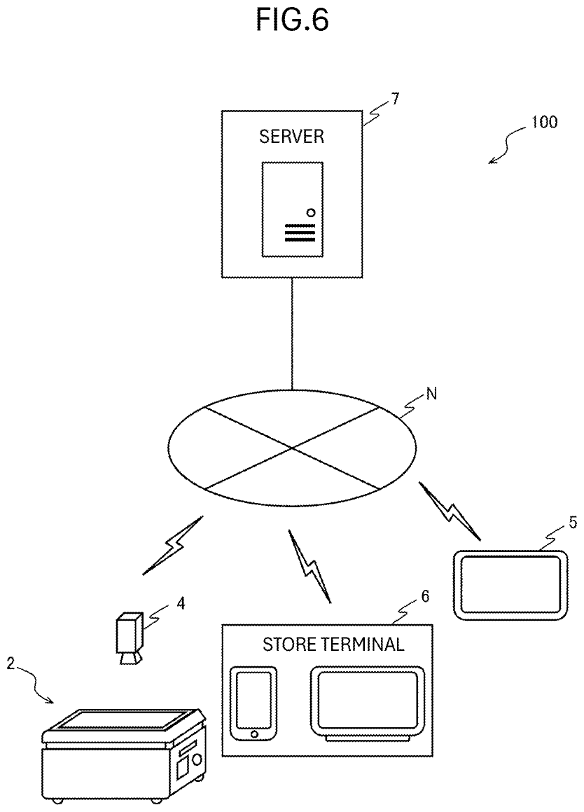

Next, referring to FIG. 6, a configuration of a deterioration prediction system 100 for the frying oil P as a frying oil deterioration prediction system will be described.

FIG. 6 illustrates a system configuration diagram of the deterioration prediction system 100 for the frying oil P.

The deterioration prediction system 100 for the frying oil P is a system for predicting the deterioration of the frying oil P and notifying a result of the prediction, and configured with, for example, store terminals 6 installed in a plurality of stores included in a convenience store chain, a supermarket chain, or the like, respectively, a server 7 that executes a program for predicting the deterioration of the frying oil P used in each of the stores, and monitors 5 (see FIG. 1) installed in the stores, respectively. The monitor 5 at each of the stores, the store terminal 6 at each of the stores, and the server 7 are directly or indirectly connected to each other via a communication network N such as Internet so as to establish the information communication therebetween.

In the deterioration prediction system 100 for the frying oil P, the store terminals 6 in the plurality of stores are configured in the same manner from each other, and thus in the following, the store terminal 6 in any of the stores is exemplified while the store terminals 6 in other stores will not be described in detail.

The store terminal 6 is an input terminal used to input information related to the frying oil P being used in a store and information related to the frying material Q, and is also a control terminal for controlling the frying oil P being used in the store. In the store terminal 6, an application for controlling of the frying oil P (hereinafter, referred to as a “frying oil control application”) is installed.

The information related to the frying oil P includes data related to measured values of the deterioration indicators of the frying oil P (for example, measured values of the acid value and those of the color). The information related to the frying material Q includes data related to the type and number of pieces of the frying material Q to be deep-fried using the frying oil P.

In the present embodiment, as illustrated in FIG. 6, the store terminal 6 is connected to the camera 4 installed in the cooking area 1 within the store so as to be able to communicate therewith, which allows the store terminal 6 to acquire the data about an image captured by the camera 4 and thus obtain a measured value of the color of the frying oil P within the oil vat 21 of the fryer 2 based on the acquired image. However, the store terminal 6 and the camera 4 do not necessarily have to be connected with each other for communication. In the case where the store terminal 6 and the camera 4 are not connected with each other for communication, the store terminal 6 acquires the data about an image captured by the camera 4 via, for example, an external device. The same is applied to acquisition of measured values of deterioration indicators other than the color in the store terminal 6.

The server 7 is an aspect of a deterioration prediction device for predicting deterioration information, which is information related to the deterioration of the frying oil P, using a measured value of a deterioration indicator of the frying oil P. In the following, the server 7 will be described as the one configured with a server device installed in a head office center or the like which manages the plurality of stores as illustrated in FIG. 6, however, it is not necessarily have to be configured with a server device. The server 7 may be configured with, for example, a cloud server or the like constructed on the communication network N.

The deterioration information includes at least one of the data indicative of the time remaining until the frying oil P reaches a predetermined oil disposal time point (remaining heating time), the data indicative of the timing at which a removing/adding oil operation is to be performed for the frying oil P in the fryer 2, the data indicative of the timing at which a filtering operation is to be performed for the frying oil P in the fryer 2, and the data indicative of the remaining number of pieces of the deep-frying material Q that can be deep-fried until the frying oil P reaches a predetermined deterioration degree.

Here, the “removing/adding oil” operation is the operation of removing a part of the frying oil P in the fryer 2 and adding (mixing) frying oil P1, which is different from the frying oil P, therein. The “frying oil P1 different from the frying oil P” does not necessarily have to be fresh oil, and may be, for example, frying oil with the deterioration degree being less than that of the frying oil P in the fryer 2. Specifically, in the case where three types of dishes, which are tempura, foods covered with bread crumbs, and karaage, are being cooked in the three units of the fryer 2, respectively, in the cooking area 1 at a store, the frying oil P in the oil vat 21 of each fryer 2 deteriorates in the order of the vat for tempura, the vat for the foods covered with bread crumbs, and the vat for karaage. In this case, for example, a part of the frying oil P in the vat for tempura or a part of that in the vat for the foods covered with bread crumbs may be mixed into the vat for karaage.

Furthermore, the “filtering” operation is the operation of passing the frying oil P in the fryer 2 through a filter to remove the pieces of foods left in the frying oil P or the like, or passing the frying oil P in the fryer 2 through a material for filtering to clean the frying oil P to make it nearly fresh.

As described above, even if the frying oil P with the deterioration degree being progressed reaches the oil disposal time point, it does not necessarily have to be wasted completely or replaced with fresh oil, but the removing/adding oil operation or the filtering operation may be performed therefor before it reaches the oil disposal time point.

The server 7 carries out the deterioration prediction processing by predicting the deterioration information about the frying oil P based on a measured value of a deterioration indicator of the frying oil P, a deterioration characteristic model indicative of the transition of the deterioration indicator of the frying oil P, and a deterioration reference value indicative of a criterion for the deterioration indicator of the frying oil P, and outputting a result of the prediction to monitor 5.

The deterioration characteristic model of the frying oil P includes at least one of the following models: a model indicative of the correlation between the heating time of the frying oil P and the deterioration indicator of the frying oil P; and a model indicative of the correlation between the number of pieces of the deep-frying material Q that can be deep-fried using the frying oil P and the deterioration indicator of the frying oil P. That is, the deterioration characteristic model of the frying oil P corresponds to a graph showing a deterioration characteristic of the frying oil P illustrated in FIG. 2 to FIG. 5 and is capable of predicting the deterioration of the frying oil P, which thus allows it to be referred to as a “deterioration prediction model” for the frying oil P.

As mentioned above, even among the frying oils P used within the same store, how the deterioration indicators thereof change is different from each other depending on the usage environment and conditions. Therefore, the server 7 is configured, not to predict the deterioration information about the frying oil P using a preset single deterioration characteristic model, but rather appropriately update the deterioration characteristic model and predict the deterioration information about the frying oil P using the updated deterioration characteristic model.

The deterioration criterion value includes at least one of an oil disposal criterion value serving as a criterion of the deterioration indicator of the frying oil P at a predetermined oil disposal time point, a removing/adding oil criterion value serving as a criterion of the deterioration indicator of the frying oil P in the fryer 2 in the case where a removing/adding oil operation needs to be performed for the frying oil P, and a filtering criterion value serving as a criterion of the deterioration indicator of the frying oil P at the time of filtering the frying oil P in the fryer 2.

These deterioration criterion values are set according to the specifications of the deep-fry cooking to be performed at the store. They may be set based on the information related to the content of the deep-fry cooking entered into the store terminal 6, or may be set to any values by a store staff, and the like.

The monitor 5 is an aspect of a notification device for notifying the deterioration information about the frying oil P predicted by the server 7. For example, upon prediction of the remaining time until the frying oil P reaches a predetermined oil disposal time point carried out by the server 7, the monitor 5 displays a message such as “00 hours left until oil disposal”. Upon prediction of the remaining number of pieces of the deep-frying material Q that can be deep-fried until the frying oil P reaches the oil disposal time point, the monitor 5 displays a message such as “you can deep-fry ΔΔ pieces until oil disposal”.

In the present embodiment, the monitor 5 is used to notify the deterioration information of the frying oil P, however, how notification is to be provided is not limited thereto, and the store terminal 6 may be used therefor. Furthermore, how the deterioration information about the frying oil P is to be notified is not limited to the one by means of displaying text, but may be performed, for example, using voices.

### (Configuration of Server 7)

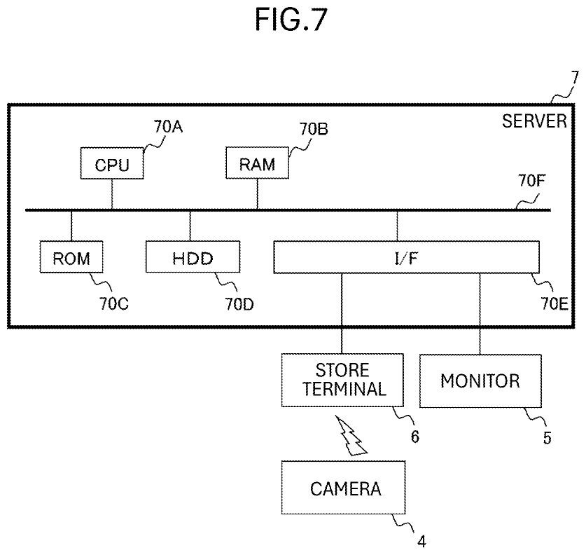

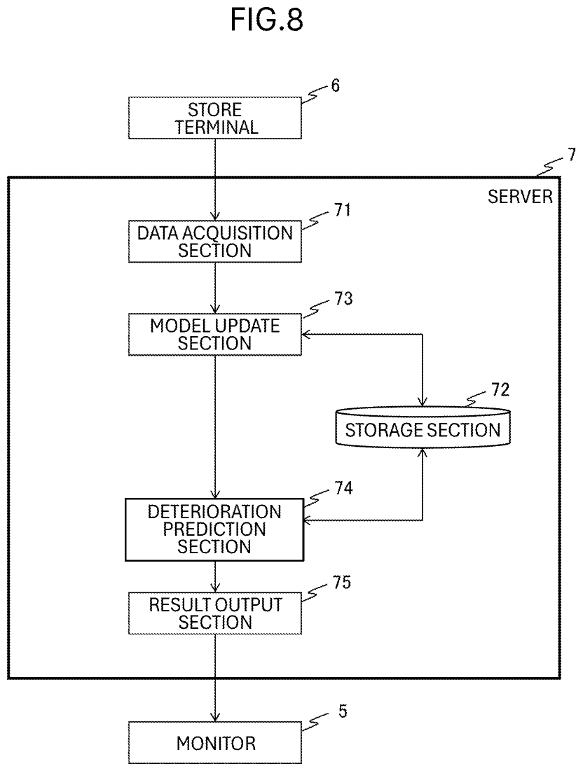

Next, referring to FIG. 7 and FIG. 8, a configuration of the server 7 will be described.

FIG. 7 illustrates an example of a hardware configuration of the server 7.

The server 7 includes a CPU (Central Processing Unit) 70A, a RAM (Random Access Memory) 70B, a ROM (Read Only Memory) 70C, an HDD (Hard Disk Drive) 70D, and an I/F (Interface) 70E, as a hardware configuration of a server device. These components are connected to each other via a common-bus 70F.

The CPU 70A is an arithmetic means and controls the whole operations of the server 7.

The RAM 70B is a volatile storage medium capable of reading and writing information at high speed, and is used, for example, as a working area when the CPU 70A processes image information.

The ROM 70C is a read-only non-volatile storage medium, and retains programs such as firmware.

The HDD 70D is a non-volatile storage medium capable of reading and writing information, and has a large storage capacity in which an OS (Operating System) and control programs and application programs for executing various kinds of information processing, which will be described later, are stored.

Any type of device such as an SSD (Solid State Drive) may be used instead of the HDD 70D as long as it realizes the functions of storing and managing information as a non-volatile storage medium.

The I/F 70E is a connection interface for connection to the communication network N, to which each of the monitors 5 and each of the store terminals 6 or the like is connected.

The server 7 including the hardware configuration described above is an information processing device for implementing the processing functions of the control program stored in the ROM 70C, the control program and application program loaded onto the RAM 70B from a storage medium such as the HDD 70D, by means of an arithmetic function provided in the CPU 70A.

By executing the information processing, a software control section including various function modules in the deterioration information processing server 7 are implemented. The functional block that realizes the functions of the server 7 is configured with a combination of the software control section thus configured and the hardware resources including the configuration described above.

In the case of the server 7 is configured with a cloud server, the hardware configuration described above is provided in a computer for implementing the cloud server (for example, a computer owned by a company which provides a cloud system or the like).

FIG. 8 is a functional block diagram illustrating the functions provided in the server 7.

The server 7 includes a data acquisition section 71, a storage section 72, a model update section 73, a deterioration prediction section 74, and a result output section 75.

The data acquisition section 71 is configured to acquire data related to a measured value of a deterioration indicator of the frying oil P output from the store terminal 6. Here, in typical cases, the deterioration indicator of the frying oil P in use is often measured in a store at a predetermined time of the day, such as after closing. Therefore, the data acquisition section 71 acquires the measured value of the deterioration indicator of the frying oil P at predetermined intervals (for example, once every twelve hours or once every twenty-four hours).

In the present embodiment, a measured value of the deterioration indicator of the frying oil P acquired by the data acquisition section 71 is the one output from the store terminal 6 to the server 7, however, it is not limited thereto, and may be data output from a measurement device for measuring the deterioration indicator of the frying oil P to the server 7.

The storage section 72 is configured to retain a deterioration characteristic model of the frying oil P and a deterioration reference value of the frying oil P. Furthermore, when the data acquisition section 71 acquires a measured value of the deterioration indicator of the frying oil P, the storage section 72 retains the measured value. In other words, the storage section 72 stores therein a previously measured value of the deterioration indicator of the frying oil P.

The model update section 73 is configured to update a deterioration characteristic model of the frying oil P stored in the storage section 72 based on a plurality of the most recent measured values of the deterioration indicator of the frying oil P, including the latest measured value of the deterioration indicator of the frying oil P acquired by the data acquisition section 71.

Specifically, the model update section 73 preferably updates a deterioration characteristic model of the frying oil P stored in the storage section 72 based on the measured values of the deterioration indicator of the frying oil P for the last two operating days of a store (which uses the frying oil P), including the latest measured value of the deterioration indicator of the frying oil P acquired by the data acquisition section 71.

This allows the storage section 72 to always retain an updated deterioration characteristic model of the frying oil P which has been updated by the model update section 73.

The deterioration prediction section 74 is configured to predict at least one of the remaining heating time until the frying oil P reaches a predetermined oil disposal time point, the timing at which a removing/adding oil operation is to be performed for the frying oil P in the fryer 2, the timing at which a filtering operation is to be performed for the frying oil P in the fryer 2, and the remaining number of pieces of the deep-frying material Q that can be deep-fried until the frying oil P reaches a predetermined deterioration degree, based on the latest measured value of the deterioration indicator of the frying oil P acquired by the data acquisition section 71, the updated deterioration characteristic model of the frying oil P updated by the model update section 73, and the deterioration reference value of the frying oil P stored in the storage section 72.

The result output section 75 is configured to output, to the monitor 5, a result of the prediction of the deterioration information about the frying oil P predicted by the deterioration prediction section 74.

### (Processing to be Executed in Server 7)

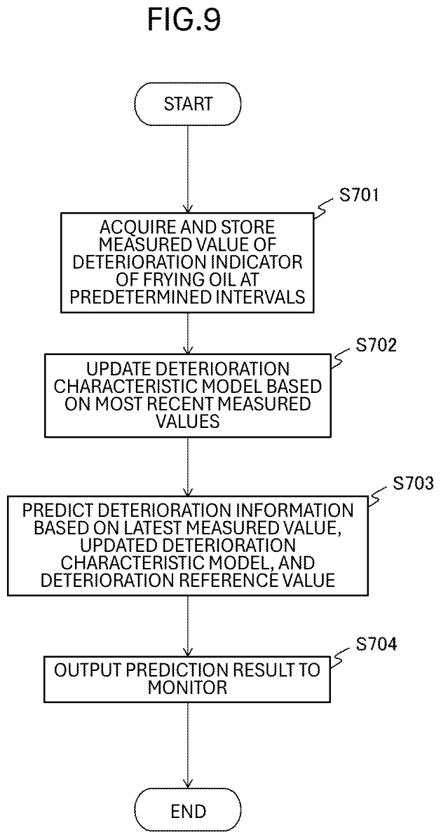

Next, referring to FIG. 9, the processing to be executed in the server 7 will be described.

FIG. 9 illustrates a flowchart of a flow of the processing to be executed in the server 7.

In the server 7, as illustrated in FIG. 9, firstly, the data acquisition section 71 acquires, at predetermined intervals, a measured value of a deterioration indicator of the frying oil P (data acquiring step), which was measured by a measurement device (for example, camera 4 in the case of using the color as the deterioration indicator) (measuring step) and output from the store terminal 6.

Every time the data acquisition section 71 acquires a measured value of the deterioration indicator of the frying oil P, the storage section 72 stores therein the measured value (step S701).

Next, the model update section 73 updates a deterioration characteristic model of the frying oil P stored in the storage section 72, based on a plurality of the most recent measured values of the deterioration indicator of the frying oil P acquired in step S701 (preferably, measured values for the last two operating days of a store, including the latest measured value) (step S702; model updating step).

Next, the deterioration prediction section 74 predicts the deterioration information about the frying oil P based on the latest measured value of the deterioration indicator of the frying oil P acquired in step S701, the updated deterioration characteristic model of the frying oil P updated in step S702, and the deterioration reference value of the frying oil P stored in the storage section 72 (step S703; deterioration predicting step).

Then, the result output section 75 outputs a result of the prediction of the deterioration information about the frying oil P predicted in step S703 to the monitor 5 (step S704; result outputting step), and then the processing in the server 7 is finished. In response to output of the result of the prediction of the deterioration information about the frying oil P from the result output section 75 (server 7), the monitor 5 notifies a staff of the store of the information about the result of the prediction by means of displaying messages or outputting voices (for example, “00 hours left until oil disposal”, “you can deep-fry ΔΔ pieces until oil disposal”, “please perform a removing/adding oil operation in ⋄⋄ hours”, and the like).

### (Update of Deterioration Characteristic Model and Prediction of Deterioration for Frying Oil P)

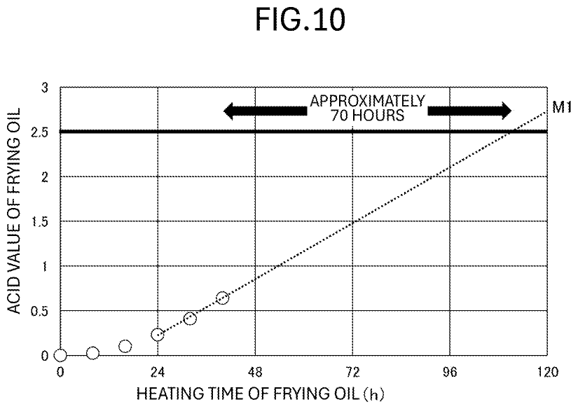

Next, referring to FIG. 10 to FIG. 18, a specific method of updating a deterioration characteristic model and predicting deterioration to be carried out in the server 7 for the frying oil P will be described.

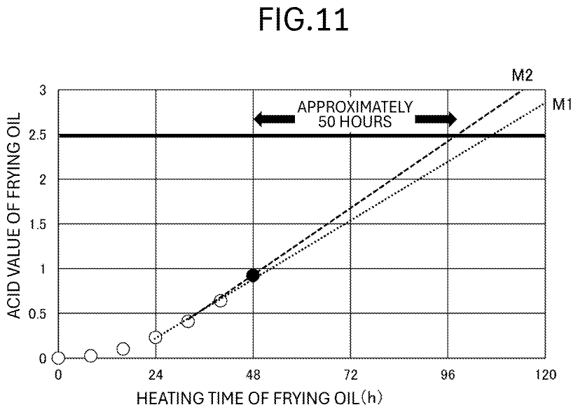

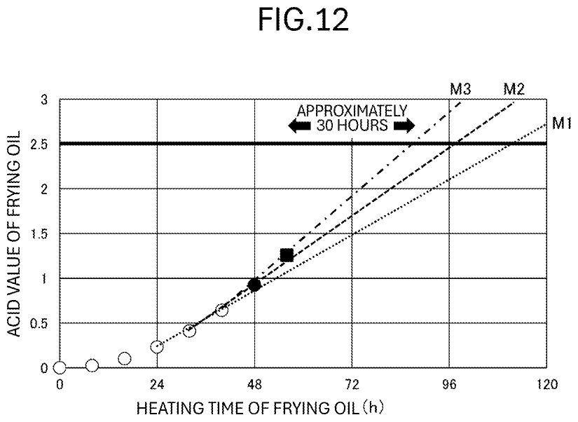

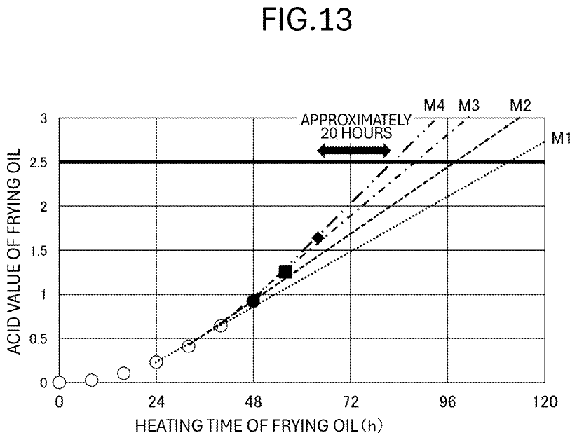

FIG. 10 illustrates an example of a deterioration characteristic model when the heating time of the frying oil P is 40 hours. FIG. 11 illustrates an update image of the deterioration characteristic model when the heating time of the frying oil P is 48 hours. FIG. 12 illustrates an update image of the deterioration characteristic model when the heating time of the frying oil P is 56 hours. FIG. 13 illustrates an update image of the deterioration characteristic model when the heating time of the frying oil P is 64 hours.

Each of FIG. 10 to FIG. 13 shows an example of a relation between the heating time of the frying oil P and the measured acid value of the frying oil P at a store where the acid value of the frying oil P is measured once every 8 hours.

As illustrated in FIG. 10, for example, when the heating time of the frying oil P reaches 40 hours, a deterioration characteristic model M1 of the frying oil P (indicated with a dotted line in FIG. 10) is expressed with a simple regression model based on the measured acid value of the frying oil P when the heating time of the frying oil P is 40 hours, that is, the latest measured acid value of the frying oil P (=approximately 0.6), the measured acid value of the frying oil P when the heating time of the frying oil P is 32 hours (=approximately 0.4), and the measured acid value of the frying oil P when the heating time of the frying oil P is 24 hours (=approximately 0.25).

In order to avoid confusion with the deterioration characteristic models M2, M3, M4 obtained by updating the deterioration characteristic model M1 by the model update section 73, in the following, the deterioration characteristic model M1 of the frying oil P when the heating time of the frying oil P reaches 40 hours is referred to as the “first deterioration characteristic model M1” for convenience of explanation.

Here, in the case where the oil disposal reference value of the frying oil P is set to 2.5, the deterioration prediction section 74 calculates that the heating time of the frying oil P at the oil disposal reference value of 2.5 is approximately 110 hours, by applying the oil disposal reference value of 2.5 to the first deterioration characteristic model M1. Then, the deterioration prediction section 74 predicts that the remaining time until the frying oil P reaches the oil disposal time point is approximately 70 hours, by subtracting the current heating time of the frying oil P (=40 hours) from the heating time of the frying oil P at the oil disposal reference value of 2.5 (=approximately 110 hours) (approximately 110 hours-40 hours).

The measurement of the deterioration indicator of the frying oil P at a store is not necessarily performed regularly (every 8 hours in FIG. 10 to FIG. 13). Therefore, the deterioration prediction section 74 calculates the current heating time of the frying oil P by applying the measured value of the deterioration indicator of the frying oil P acquired by the data acquisition section 71 to the deterioration characteristic model of the frying oil P.

Subsequently, as illustrated in FIG. 11, when the heating time of the frying oil P reaches 48 hours, for predicting the remaining time until the frying oil P reaches the oil disposal time point, the deterioration prediction section 74 does not use the first deterioration characteristic model M1 (indicated with a dotted line in FIG. 11) stored in the memory section 72, but uses an updated deterioration characteristic model M2 (indicated with a dashed line in FIG. 11) obtained by updating the first deterioration characteristic model M1 by the model update section 73.

In the following, the deterioration characteristic model M2 of the frying oil P obtained by updating the first deterioration characteristic model M1 by the model update section 73 is referred to as the “second deterioration characteristic model M2” for convenience of explanation.

The second deterioration characteristic model M2 is expressed with a simple regression model based on the measured acid value of the frying oil P when the heating time of the frying oil P is 48 hours, that is, the latest measured acid value of the frying oil P (=approximately 0.9), the measured acid value of the frying oil P when the heating time of the frying oil P is 40 hours (=approximately 0.6), and the measured acid value of the frying oil P when the heating time of the frying oil P is 32 hours (=approximately 0.4).

As illustrated in FIG. 11, in the second deterioration characteristic model M2, the latest measured acid value of the frying oil P (=approximately 0.9) is reflected, which makes a slope thereof steeper than that of the first deterioration characteristic model M1. Thus, the second deterioration characteristic model M2 is more consistent with the actual deterioration progress of the frying oil P at the current time (when the heating time of the frying oil P reaches 48 hours).

The deterioration prediction section 74 predicts that the remaining time until the frying oil P reaches the oil disposal time point is approximately 50 hours, by applying the latest measured acid value of the frying oil P (=approximately 0.9) acquired by the data acquisition section 71 and the oil disposal reference value of 2.5 stored in the storage section 72 to the updated second deterioration characteristic model M2 updated by model update section 73, respectively.

Here, if applying the latest measured acid value of the frying oil P (=approximately 0.9) acquired by the data acquisition section 71 and the oil disposal reference value of 2.5 stored in the storage section 72 to the first deterioration characteristic model M1 which is the deterioration characteristic model before being updated, respectively, the remaining time until the frying oil P reaches the oil disposal time point is calculated as approximately 62 hours, which is different from the result of prediction when applying the values to the second deterioration characteristic model M2.

Subsequently, as illustrated in FIG. 12, for predicting the remaining time until the frying oil P reaches the oil disposal time point when the heating time of the frying oil P reaches 56 hours, the deterioration prediction section 74 does not use the second deterioration characteristic model M2 (indicated with a dashed line in FIG. 12) stored in the memory section 72, but uses an updated deterioration characteristic model M3 (indicated with a chain line in FIG. 12) obtained by updating the second deterioration characteristic model M2 by the model update section 73.

In the following, the deterioration characteristic model M3 obtained by updating the second deterioration characteristic model M2 by the model update section 73 is referred to as the “third deterioration characteristic model M3” for convenience of explanation.

The third deterioration characteristic model M3 is expressed with a simple regression model based on the measured acid value of the frying oil P when the heating time of the frying oil P is 56 hours, that is, the latest measured acid value of the frying oil P (=approximately 1.3), the measured acid value of the frying oil P when the heating time of the frying oil P is 48 hours (=approximately 0.9), and the measured acid value of the frying oil P when the heating time of the frying oil P is 40 hours (=approximately 0.6).

As illustrated in FIG. 12, in the third deterioration characteristic model M3, the latest measured acid value of the frying oil P (=approximately 1.3) is reflected, which makes a slope thereof steeper than that of the first deterioration characteristic model M1 and that of the second deterioration characteristic model M2. Thus, the third deterioration characteristic model M3 is more consistent with the actual deterioration progress of the frying oil P at the current time (when the heating time of the frying oil P is 56 hours).

The deterioration prediction section 74 predicts that the remaining time until the frying oil P reaches the oil disposal time point is approximately 30 hours, by applying the latest measured acid value of the frying oil P (=approximately 1.3) acquired by the data acquisition section 71 and the oil disposal reference value of 2.5 stored in the storage section 72 to the updated third deterioration characteristic model M3 updated by the model update section 73, respectively.

Here, if applying the latest measured acid value of the frying oil P (=approximately 1.3) acquired by the data acquisition section 71 and the oil disposal reference value of 2.5 stored in the storage section 72 to the second deterioration characteristic model M2 which is the deterioration characteristic model before being updated, respectively, the remaining time until the frying oil P reaches the oil disposal time point is calculated as approximately 42 hours, which is different from the result of prediction when applying the values to the third deterioration characteristic model M3.

Furthermore, if applying the latest measured acid value of the frying oil P (=approximately 1.3) acquired by the data acquisition section 71 and the oil disposal reference value of 2.5 stored in the storage section 72 to the first deterioration characteristic model M1 which is the model when the heating time of the frying oil P is 40 hours, respectively, the remaining time until the frying oil P reaches the oil disposal time point is calculated as approximately 54 hours, which is much more different from the result of prediction when applying the values to the third deterioration characteristic model M3.

Subsequently, as illustrated in FIG. 13, for predicting the remaining time until the frying oil P reaches the oil disposal time point when the heating time of the frying oil P reaches 64 hours, the deterioration prediction section 74 does not use the third deterioration characteristic model M3 (indicated with a chain line in FIG. 13) stored in the memory section 72, but uses an updated deterioration characteristic model M4 (indicated with a double-chain line in FIG. 13) obtained by updating the third deterioration characteristic model M3 by the model update section 73.

In the following, the deterioration characteristic model M4 obtained by updating the third deterioration characteristic model M3 by the model update section 73 is referred to as the “fourth deterioration characteristic model M4” for convenience of explanation.

The fourth deterioration characteristic model M4 is expressed with a simple regression model based on the measured acid value of the frying oil P when the heating time of the frying oil P is 64 hours, that is, the latest measured acid value of the frying oil P (=approximately 1.7), the measured acid value of the frying oil P when the heating time of the frying oil P is 56 hours (=approximately 1.3), and the measured acid value of the frying oil P when the heating time of the frying oil P is 48 hours (=approximately 0.9).

As illustrated in FIG. 13, in the fourth deterioration characteristic model M4, the latest measured acid value of the frying oil P (=approximately 1.7) is reflected, which makes a slope thereof steeper than that of the first deterioration characteristic model M1, that of the second deterioration characteristic model M2, and that of the third deterioration characteristic model M3. Thus, the fourth deterioration characteristic model M4 is more consistent with the actual deterioration progress of the frying oil P at the current time (when the heating time of the frying oil P is 64 hours).

The deterioration prediction section 74 predicts that the remaining time until the frying oil P reaches the oil disposal time point is approximately 20 hours, by applying the latest measured acid value of the frying oil P (=approximately 1.7) acquired by the data acquisition section 71 and the oil disposal reference value of 2.5 stored in the storage section 72 to the updated fourth deterioration characteristic model M4 updated by the model update section 73, respectively.

Here, if applying the latest measured acid value of the frying oil P (=approximately 1.7) acquired by the data acquisition section 71 and the oil disposal reference value of 2.5 stored in the storage section 72 to the third deterioration characteristic model M3 which is the deterioration characteristic model before being updated, respectively, the remaining time until the frying oil P reaches the oil disposal time point is calculated as approximately 22 hours, which is different from the result of prediction when applying the values to the fourth deterioration characteristic model M4.

Furthermore, if applying the latest measured acid value of the frying oil P (=approximately 1.7) acquired by the data acquisition section 71 and the oil disposal reference value of 2.5 stored in the storage section 72 to the second deterioration characteristic model M2 which is the model when the heating time of the frying oil P is 48 hours, respectively, the remaining time until the frying oil P reaches the oil disposal time point is calculated as approximately 26 hours, which is much more different from the result of prediction when applying the values to the fourth deterioration characteristic model M4.

Still further, if applying the latest measured acid value of the frying oil P (=approximately 1.7) acquired by the data acquisition section 71 and the oil disposal reference value of 2.5 stored in the storage section 72 to the first deterioration characteristic model M1 which is the model when the heating time of the frying oil P is 40 hours, respectively, the remaining time until the frying oil P reaches the oil disposal time point is calculated as approximately 30 hours, which is much more different from the result of prediction when applying the values to the fourth deterioration characteristic model M4.

As described above, the server 7 updates a deterioration characteristic model of the frying oil P based on a plurality of most recent measured values of a deterioration indicator, including the latest measured value of the deterioration indicator of the frying oil P, and predicts the deterioration information about the frying oil P using the updated deterioration characteristic model. Therefore, according to the present embodiment, a result of prediction in which the actual deterioration progress of the frying oil P is reflected can be obtained even if the usage environment or usage conditions of the frying oil P change. This enables a result of prediction with more accuracy to be obtained in the server 7, compared to the case of predicting the deterioration of the frying oil P using a predetermined deterioration characteristic model.

As described above, the present invention allows a store using the frying oil P to avoid the possibilities that the frying oil P that can still be used is wasted or the frying oil P that has already passed the time for disposal is still be used. Therefore, the present invention can contribute to the efforts to promote the Sustainable Development Goals (2030 Agenda for Sustainable Development, adopted by the United Nations on Sep. 25, 2015, hereinafter, referred as “SDGs”).

The method of predicting the deterioration of the frying oil P in server 7 is applicable to the prediction of the timing of performing a removing/adding oil operation for the frying oil P in the fryer 2, the timing of performing a filtering operation for the frying oil P in the fryer 2, and the remaining number of pieces of the deep-frying material that can be deep-fried until the frying oil P reaches a predetermined deterioration degree, in the same manner.

For convenience of explanation, the example using the first deterioration characteristic model M1, the second deterioration characteristic model M2, the third deterioration characteristic model M3, and the fourth deterioration characteristic model M4 as the deterioration characteristic models of the frying oil P has been described, however, the server 7 is not configured to store a plurality of deterioration characteristic models (first deterioration characteristic model M1, second deterioration characteristic model M2, third deterioration characteristic model M3, and fourth deterioration characteristic model M4) in the storage section 72 and select and use one of the deterioration characteristic models in prediction of the deterioration information about the frying oil P. It should be noted that, in the present invention, the server 7 is configured to appropriately update a single deterioration characteristic model and use the updated deterioration characteristic model in prediction of the deterioration information about the frying oil P.

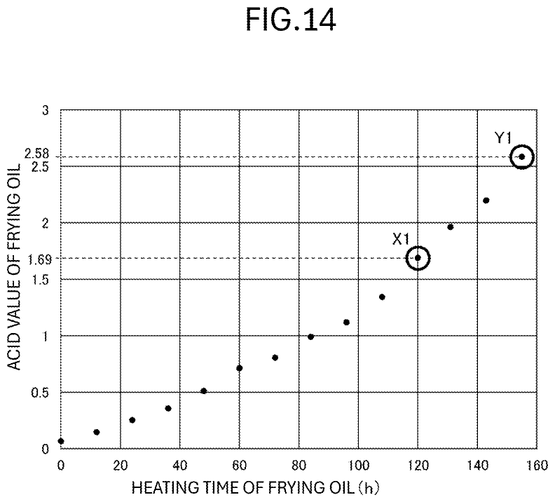

It is preferable that the server 7 uses measured values of a deterioration indicator of the frying oil P for the last two operating days of a store, including the latest measured value of the deterioration indicator of the frying oil P as acquired, when updating a deterioration characteristic model of the frying oil P. In the following, referring to FIG. 14 to FIG. 18, operations and effects of this case will be described.

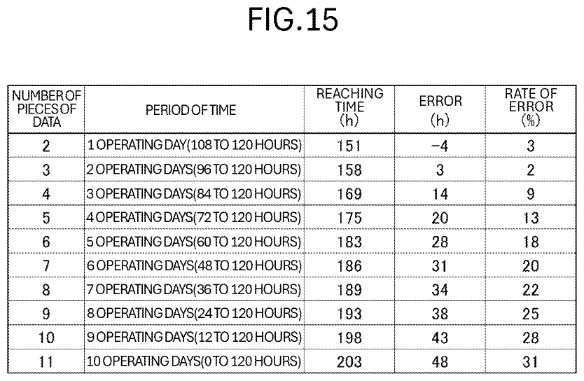

FIG. 14 illustrates how the acid value of the frying oil P changes relative to the heating time of the frying oil P at a supermarket store. FIG. 15 illustrates a table for the graph illustrated in FIG. 14, in which the number of pieces of data used for a deterioration characteristic model, a period of time corresponding to the number of pieces of data, the time required to reach an oil disposal time point from the current time, an error, and the rate of error are listed.

The operating hours of this supermarket store are 12 hours a day, and a staff of the store measures the acid value (deterioration indicator) of the frying oil P once a day (one operating day). In FIG. 14, an interval between two adjacent plots represents one operating day.

Here, an example in which, when the heating time of the frying oil P reaches 120 hours, the server 7 predicts the remaining time until the frying oil P reaches an oil disposal reference value of 2.58 (plot indicated with Y1 in FIG. 14) based on each of the measured acid values of the frying oil P until it reaches the measured acid value of 1.69 (plot indicated with X1 in FIG. 14) when the heating time of the frying oil P is 120 hours, among the measured acid values of the frying oil P illustrated in FIG. 14, will be described.

As illustrated in FIG. 15, in the case where the server 7 firstly uses a deterioration characteristic model of the frying oil P which is based on measured acid values of the frying oil P for the most recent one operating day (in other words, two pieces of data when the heating time of the frying oil P reaches 108 hours and 120 hours, respectively), including the latest measured acid value (=1.69) of the frying oil P, the remaining time until the frying oil P reaches the oil disposal time point (hereinafter, simply referred to as “reaching time”) is 151 hours. Thus, the rate of error between this reaching time and the actual reaching time is 3% (error is-4 hours).

Next, in the case where the server 7 uses a deterioration characteristic model of the frying oil P which is based on measured acid values of the frying oil P for the most recent two operating days (in other words, three pieces of data when the heating time of the frying oil P reaches 96 hours, 108 hours, and 120 hours, respectively), including the latest measured acid value (=1.69) of the frying oil P, the reaching time is 158 hours. Thus, the rate of error between this reaching time and the actual reaching time is 2% (error is 3 hours).

Then, in the case where the server 7 uses a deterioration characteristic model of the frying oil P which is based on measured acid values of the frying oil P for the most recent three operating days (in other words, four pieces of data when the heating time of the frying oil P reaches 84 hours, 96 hours, 108 hours, and 120 hours, respectively), including the latest measured acid value (=1.69) of the frying oil P, the reaching time is 169 hours. Thus, the rate of error between this reaching time and the actual reaching time is 9% (error is 14 hours).

Thus, in the case where the server 7 uses a deterioration characteristic model of the frying oil P which is based on measured acid values of the frying oil P for the most recent three operating days, the rate of error between the reaching time predicted by the server 7 and the actual reaching time highly increases compared to the case of using a deterioration characteristic model of the frying oil P which is based on measured acid values of the frying oil P for the most recent one operating day and the case of using that for the most recent two operating days.

Furthermore, as the pieces of data about measured acid values of the frying oil P which serve as the basis for a deterioration characteristic model of the frying oil P (in FIG. 15, in the case where the number of pieces of data is 4 or more, that is, three operating days and thereafter) increases, the rate of error between the reaching time predicted by the server 7 and the actual reaching time gradually increase.

Therefore, it can be said that this supermarket store can improve the accuracy of prediction when the server 7 predicts the deterioration information about the frying oil P using a deterioration characteristic model of the frying oil P updated based on measured acid values of the frying oil P for the most recent one operating day or two operating days, which are acquired by the data acquisition section 71.

Next, an example in which intervals for measuring the acid value of the frying oil P differ from those illustrated in FIG. 14 and FIG. 15 will be described.

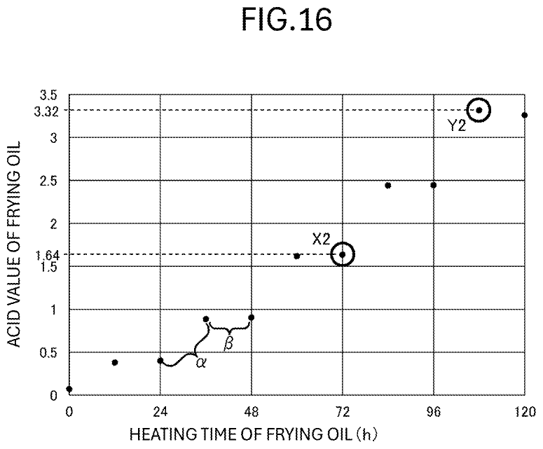

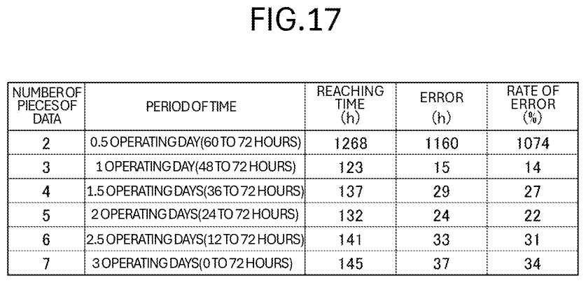

FIG. 16 illustrates how the acid value of the frying oil P changes relative to the heating time of the frying oil P at a convenience store. FIG. 17 illustrates a table for the graph illustrated in FIG. 16, in which the number of pieces of data used for a deterioration characteristic model, a period of time corresponding to the number of pieces of data, the time required to reach an oil disposal time point from the current time, an error, and the rate of error are listed.

The operating hours of this convenience store are 24 hours a day, and a staff of the store measures the acid value of the frying oil P twice a day (one operating day). In FIG. 16, an interval between the two ends of a set of three consecutive plots represents one operating day, in other words, an interval between two adjacent plots represents 0.5 operating day.

At this convenience store, one operating day includes two types of time zones, that is, one in which deep-fry cooking is frequently performed (for example, time zone indicated with α in FIG. 16) and another in which empty heating is frequently performed (for example, time zone indicated with β in FIG. 16). Here, “empty heating” refers to heating only the frying oil P without cooking any deep-frying material Q (ingredient). As illustrated in FIG. 16, the frying oil P deteriorates less during empty heating than during deep-fry cooking.

In the following, when the heating time of the frying oil P reaches 72 hours, the server 7 predicts the remaining time until the frying oil P reaches an oil disposal reference value of 3.32 (plot indicated with Y2 in FIG. 16) based on each of the measured acid values of the frying oil P until it reaches the measured acid value of 1.64 (plot indicated with X2 in FIG. 16) when the heating time of the frying oil P is 72 hours, among the measured acid values of the frying oil P illustrated in FIG. 16, will be described.

As illustrated in FIG. 17, in the case where the server 7 uses a deterioration characteristic model of the frying oil P which is based on measured acid values of the frying oil P for the most recent 0.5 operating day (in other words, two pieces of data when the heating time of the frying oil P reaches 60 hours and 72 hours, respectively), including the latest measured acid value (=1.64) of the frying oil P, the reaching time is 1268 hours. Thus, the rate of error between this reaching time and the actual reaching time is 1074% (error is 1160 hours).

In this case, no deep-fry cooking was performed between the two pieces of data which serve as the basis for a deterioration characteristic model of the frying oil P, and only the influence of the empty heating was reflected in the deterioration characteristic model of the frying oil P. As a result, the rate of error between the reaching time predicted by the server 7 and the actual reaching time has become extremely large.

On the other hand, in the case where the server 7 uses a deterioration characteristic model of the frying oil P which is based on measured acid values of the frying oil P for the most recent one operating day (in other words, three pieces of data when the heating time of the frying oil P reaches 48 hours, 60 hours, and 72 hours, respectively), including the latest measured acid value (=1.64) of the frying oil P, the reaching time is 123 hours. Thus, the rate of error between this reaching time and the actual reaching time is 14% (error is 15 hours).

In this case, both the influence of the deep-fry cooking and that of the empty heating are reflected in the deterioration characteristic model of the frying oil P. This enables reduction in the rate of error between the reaching time predicted by the server 7 and the actual reaching time and thus improvement in the accuracy of prediction by the server 7.

In the case where the server 7 uses a deterioration characteristic model of the frying oil P which is based on measured acid values of the frying oil P for the most recent 1.5 operating days (in other words, four pieces of data when the heating time of the frying oil P reaches 36 hours, 48 hours, 60 hours, and 72 hours, respectively), including the latest measured acid value (=1.64) of the frying oil P, the reaching time is 137 hours. Thus, the rate of error between this reaching time and the actual reaching time is 27% (error is 29 hours).

In the same manner as the case of a supermarket store, as the pieces of data about measured acid values of the frying oil P which serve as the basis for a deterioration characteristic model of the frying oil P increase, the rate of error between the reaching time predicted by the server 7 and the actual reaching time gradually increase.

Therefore, it can be said that this convenience store can improve the accuracy of prediction when the server 7 predicts the deterioration information about the frying oil P using a deterioration characteristic model of the frying oil P updated based on measured acid values of the frying oil P for the most recent one operating day, which are acquired by the data acquisition section 71.

As described above, considering the contents illustrated in FIG. 15 and FIG. 17, it can be said that the most accurate prediction can be realized when the server 7 predicts the deterioration information about the frying oil P using a deterioration characteristic model of the frying oil P updated based on measured acid values of the frying oil P for the most recent one operating day, which are acquired by the data acquisition section 71.

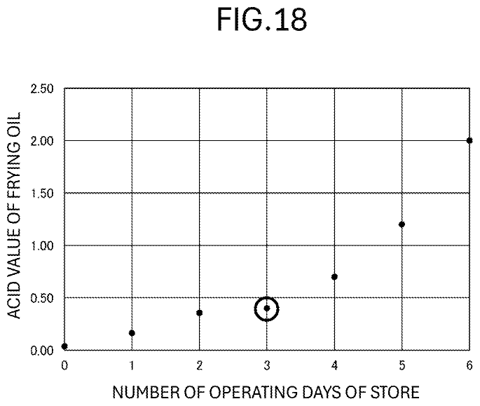

FIG. 18 illustrate how the acid value of the frying oil P changes relative to the number of operating days of a store.

As illustrated in FIG. 18, at this store, the measured acid value of the frying oil P is approximately 0.2 when one operating day passes after the frying oil P in a fresh condition starts to be used, the measured acid value of the frying oil P is approximately 0.4 when two operating days pass, which shows that the frying oil P gradually deteriorates. In typical cases, thereafter, the measured acid value of the frying oil P increases at a predetermined rate as it passes to third, fourth, fifth, and sixth operating days.

However, in FIG. 18, when three operating days passes after the frying oil P in the fresh condition starts to be used, the measured acid value of the frying oil P is approximately 0.45, which is not significantly different from the acid value of the frying oil P measured after the second operating day (approximately 0.4) (indicated with a circle in FIG. 18). This is because the sales of fried foods on the third operating day were less than those on the other operating days and thus the frying oil P hardly deteriorated.

Here, in the case where the server 7 predicts the deterioration information about the frying oil P using a deterioration characteristic model of the frying oil P updated based on the measured acid values of the frying oil P for the most recent one operating day acquired by the data acquisition section 71, if the measured acid values of the frying oil P for one day on the third operating day illustrated in FIG. 18 are used, the prediction will be influenced only by the deterioration of the frying oil P on that day (third operating day illustrated in FIG. 18). This causes a difference from the result of prediction of the deterioration information about the frying oil P when using the measured acid values of the frying oil P for one day of each of the other operating days illustrated in FIG. 18.

Thus, in the case where a deterioration characteristic model of the frying oil P is based on the measured acid values of the frying oil P acquired by the data acquisition unit 71 for the most recent one operating day, if the sales of fried foods happened to be low on that day and the deterioration of the frying oil P did not progress, the accuracy of prediction of the deterioration information about the frying oil P by the server 7 may significantly decrease.

Therefore, it is preferable that the server 7 updates a deterioration characteristic model of the frying oil P based on measured values of a deterioration indicator of the frying oil P for the last two operating days of a store, including the latest measured value of the deterioration indicator of the frying oil P acquired by the data acquisition section 71, in order to avoid decrease in the accuracy of prediction caused by fluctuations in the sales of fried food at the store.

In the above, the embodiment of the present invention has been described. However, the present invention is not limited to the embodiment described above but various modifications can be made therein. For example, the embodiment is described in detail herein for the purpose of clarity and concise description, and the present invention is not limited to those including all the features described above. Furthermore, some of the features according to a predetermined embodiment can be replaced with other features according to a separate embodiment, and other features can be added to the configuration of the predetermined embodiment. Still further, other features of a separate embodiment may be added to some of the features of each of the embodiments described above, and some of the features of each of the embodiments described above may be deleted or replaced.

For example, in the embodiment described above, the server 7 has been described as an aspect of the deterioration prediction device for the frying oil P, however, it is not limited thereto and the functions provided in the deterioration prediction device for the frying oil P may be provided in a frying oil management application installed in the store terminal 6.

Furthermore, in the embodiment described above, the deterioration indicator of the frying oil P has been mainly described referring to the cases where it is the color or the acid value, however, it is not limited thereto. The present invention can be realized even if using a deterioration indicator other than the color or the acid value.

Still further, in the embodiment described above, the example in which the deterioration characteristic model of the frying oil P is a simple regression model expressed by a liner equation has been described, however, a deterioration characteristic model is not limited to a particular type. Other models, such as multiple regression or other linear regression models, or models generated by machine learning, may be used.

Still further, in the embodiment described above, the example in which oil and fat is the frying oil P has been described. However, the oil and fat to which the present invention is applied is not necessarily the edible oil to be used for deep-fry cooking, but may be edible oil to be used for other cooking purposes or other types of oils and fats (such as industrial oils).

### REFERENCE SIGNS LIST

4: camera (measurement device)
5: monitor (notification device)
6: store terminal (notification device, oil and fat deterioration prediction device)
7: server (oil and fat deterioration prediction device)
71: data acquisition section
72: storage section
73: model update section
74: deterioration prediction section
75: result output section
100: frying oil deterioration prediction system (oil and
fat deterioration prediction system)
P: frying oil (edible oil)
Q: deep-frying material (ingredient)

1. An oil and fat deterioration prediction device for predicting deterioration of oil and fat, comprising:
a storage section configured to store a deterioration characteristic model and a deterioration reference value, the deterioration characteristic model being indicative of transition of a deterioration indicator that is an indicator indicative of a deterioration degree of the oil and fat, the deterioration reference value being indicative of a reference related to the deterioration indicator;
a data acquisition section configured to acquire a measured value of the deterioration indicator;
a model update section configured to update the deterioration characteristic model stored in the storage section based on a plurality of most recent measured values acquired by the data acquisition section;
a deterioration prediction section configured to predict deterioration information that is information about deterioration of the oil and fat, based on a latest measured value acquired by the data acquisition section, the deterioration characteristic model updated by the model update section, and the deterioration reference value stored in the storage section; and
a result output section configured to output a result of prediction of the deterioration information predicted by the deterioration prediction section.

2. The oil and fat deterioration prediction device according to claim 1, wherein
the oil and fat is edible oil to be used in cooking of an ingredient using a cooking tool, and
the deterioration information includes at least one of:
data indicative of a remaining time until the edible oil reaches a predetermined oil disposal time point;
data indicative of a timing of performing a removing/adding oil operation, in which a part of the edible oil in the cooking tool is removed and another edible oil that is different from the edible oil is added therein;
data indicative of a timing of filtering the edible oil in the cooking tool; or
data indicative of a remaining amount of ingredients that are cookable until the edible oil reaches a predetermined deterioration degree.

3. The oil and fat deterioration prediction device according to claim 2, wherein
the deterioration characteristic model includes at least one of:
a model indicative of a correlation between a heating time of the edible oil and the deterioration indicator; or
a model indicative of a correlation between an amount of ingredients that are cookable using the edible oil and the deterioration indicator.

4. The oil and fat deterioration prediction device according to claim 1, wherein
the data acquisition section acquires the measured value of the deterioration indicator at predetermined intervals, and
the model update section updates the deterioration characteristic model stored in the storage section based on measured values for most recent two operating days of a store that uses the oil and fat, including the latest measured value acquired by the data acquisition section.

5. An oil and fat deterioration prediction system for predicting deterioration of oil and fat and notifying a result of prediction, the system comprising:
a measurement device for measuring a deterioration indicator that is an indicator indicative of a deterioration degree of the oil and fat;
an oil and fat deterioration prediction device for predicting deterioration information that is information about the deterioration of the oil and fat, using a measured value of the deterioration indicator measured by the measurement device; and
a notification device for notifying the result of prediction of the deterioration information predicted by the oil and fat deterioration prediction device,
the oil and fat deterioration prediction device being configured to:
store a deterioration characteristic model indicative of transition of the deterioration indicator and a deterioration reference value indicative of a reference related to the deterioration indicator;
acquire the measured value of the deterioration indicator measured by the measurement device;
update the deterioration characteristic model as stored based on a plurality of most recent measured values as acquired;
predict the deterioration information based on a latest measured value as acquired, the deterioration characteristic model as updated, and the deterioration reference value as stored; and
output the result of prediction of the deterioration information as predicted to the notification device.

6. The oil and fat deterioration prediction system according to claim 5, wherein
the oil and fat is edible oil to be used in cooking of an ingredient using a cooking tool, and
the deterioration information includes at least one of:
data indicative of a remaining time until the edible oil reaches a predetermined oil disposal time point;
data indicative of a timing of performing a removing/adding oil operation, in which a part of the edible oil in the cooking tool is removed and another edible oil that is different from the edible oil is added therein;
data indicative of a timing of filtering the edible oil in the cooking tool; or
data indicative of a remaining amount of ingredients that are cookable until the edible oil reaches a predetermined deterioration degree.

7. The oil and fat deterioration prediction system according to claim 6, wherein
the deterioration characteristic model includes at least one of:
a model indicative of a correlation between a heating time of the edible oil and the deterioration indicator; or
a model indicative of a correlation between an amount of ingredients that are cookable using the edible oil and the deterioration indicator.

8. The oil and fat deterioration prediction system according to claim 5, wherein
the oil and fat deterioration prediction device is configured to:
acquire the measured value of the deterioration indicator at predetermined intervals, and
update the deterioration characteristic model as stored, based on measured values for most recent two operating days of a store that uses the oil and fat, including the latest measured value as acquired.

9. An oil and fat deterioration prediction method for predicting deterioration of oil and fat and notifying a result of prediction, using a measurement device for measuring a deterioration indicator that is an indicator indicative of a deterioration degree of the oil and fat, an oil and fat deterioration prediction device for predicting deterioration information that is information about the deterioration of the oil and fat using a measured value of the deterioration indicator measured by the measurement device, and a notification device for notifying the result of prediction of the deterioration information predicted by the oil and fat deterioration prediction device, the oil and fat deterioration prediction device being configured to store a deterioration characteristic model indicative of transition of the deterioration indicator and a deterioration reference value indicative of a reference related to the deterioration indicator,
the method comprising:
a measuring step of measuring the deterioration indicator, by the measurement device;
a data acquiring step of acquiring the measured value measured in the measuring step, by the oil and fat deterioration prediction device;
a model updating step of updating the deterioration characteristic model as stored based on a plurality of most recent measured values acquired in the data acquiring step, by the oil and fat deterioration prediction device;
a deterioration predicting step of predicting the deterioration information based on a latest measured value acquired in the data acquiring step, the deterioration characteristic model updated in the model updating step, and the deterioration reference value as stored, by the oil and fat deterioration prediction device;
a result outputting step of outputting the result of prediction predicted in the deterioration predicting step to the notification device, by the oil and fat deterioration prediction device; and
a notifying step of acquiring and notifying the result of prediction of the deterioration information output in the result outputting step, by the notification device.

10. The oil and fat deterioration prediction method according to claim 9, wherein
the oil and fat is edible oil to be used in cooking of an ingredient using a cooking tool, and
the deterioration information includes at least one of:
data indicative of a remaining time until the edible oil reaches a predetermined oil disposal time point;
data indicative of a timing of performing a removing/adding oil operation, in which a part of the edible oil in the cooking tool is removed and another edible oil that is different from the edible oil is added therein;
data indicative of a timing of filtering the edible oil in the cooking tool; or
data indicative of a remaining amount of ingredients that are cookable until the edible oil reaches a predetermined deterioration degree.

11. The oil and fat deterioration prediction method according to claim 10, wherein
the deterioration characteristic model includes at least one of:
a model indicative of a correlation between a heating time of the edible oil and the deterioration indicator; or
a model indicative of a correlation between an amount of ingredients that are 80 cookable using the edible oil and the deterioration indicator.

12. The oil and fat deterioration prediction method according to claim 9, wherein
in the data acquiring step, the oil and fat deterioration prediction device acquires the measured value of the deterioration indicator at predetermined intervals, and
in the model updating step, the oil and fat deterioration prediction device updates the deterioration characteristic model as stored based on measured values for most recent two operating days of a store that uses the oil and fat, including the latest measured value acquired in the data acquiring step.
